(19) United States
(12) Patent Application Publication
(10) Pub. No.: US 2010/0055718 A1
(43) Pub. Date: Mar. 4, 2010

(54) NANOPLATE DYE PLATFORM AND METHODS OF MAKING AND USING THE SAME

(76) Inventor: Kwangyeol Lee, Namyangju-si (KR)

Correspondence Address: EDWARDS ANGELL PALMER & DODGE LLP, P.O. BOX 55874, BOSTON, MA, 02205, US

(21) Appl. No.: 12/202,154

(22) Filed: Aug. 29, 2008

(51) Int. Cl. G01N 33/566

(52) U.S. Cl. 435/7.21

(57) ABSTRACT

Embodiments disclosed herein relate to labeling reagents comprising a plurality of nanoplates attached to dye molecules. The nanoplates may be configured into stacks and/or at least partially surrounded by a surrounding layer. The reagent may then be used to label a target (e.g., structure or environment).

### BACKGROUND

Dye-based techniques have become a powerful tool in scientific research and clinical diagnostics, as well as in many industrial applications, for the detection of biomolecules using various assays including, but not limited to, flow cytometry, nucleic acid hybridization, DNA sequencing, nucleic acid amplification, immunoassays, histochemistry, and functional assays involving living cells. In some instances, dyes are used to detect cancerous cells or tissues.

Dyes may include fluorescent molecules, such that the location of the dye-attached or labeled substance can be visually identified. In some instances, the dye continually emits light, which may fade over time. In other instances, the dye emits light once it has absorbed energy at a particular wavelength. In still other instances, the light emitted by the dye depends on an interaction with a biomolecule. For example, the dye may emit a different wavelength depending on whether two molecules have bound together.

While dyes can be used to determine a location of a biomolecule or a quantity of the biomolecule, such assessments depend on the ability to detect the signal emitted by the dye. However, frequently the signal is too weak to be reliably detected.

### SUMMARY

In some aspects, there can be reagents that include, for example, one or more metal oxide nanoplates; and one or more dyes attached to surface of said one or more metal oxide nanoplates. The reagents further can include a plurality of said metal oxide nanoplates, and each nanoplate of the plurality can include, for example, at least one dye-attached surface. The at least one dye-attached surface can include one or more interfaces between adjacent nanoplates. For example, the interface between adjacent plates can be a layer of dye molecules. The one or more nanoplates or plurality of metal oxide nanoplates can include a stack of nanoplates. Furthermore, the reagents can include at least one polymer at least partly surrounding the one or more nanaoplates and dye(s). The polymer can be, for example, hydrophilic, hydrophobic or both hydrophilic and hydrophobic. In some aspects, the polymer can include a block co-polymer. In some aspects the metal oxide can include one or more of La₂O₃, Pr₂O₃, Nd₂O₃, Sm₂O₃, Gd₂O₃, Dy₂O₃, Ce₂O₃, Tb₂O₃, Er₂O₃, Eu₂O₃, Lu₂O₃, Tm₂O₃, Ho₂O₃, Pm₂O₃, Yb₂O₃ or the like. In some aspects the metal oxide can include lanthanide oxide La₂O₃. The reagent can be, for example, water soluble. The reagents can include an additional component, for example, one or more of a biomolecule, an antibody, an aptamer, an antigen, a monoclonal antibody, a protein, an enzyme, a receptor, a natural or synthetic drug, a synthetic polymer, a hormone, a lymphokine, a cytokine, a toxin, a ligand, a hapten, a carbohydrate, a sugar, an oligopeptide, a polypeptide, a nucleobase, a nucleic acid molecule, and a liposome.

In some aspects, there can be methods of determining a property of a target. The methods can include, for example, introducing a metal oxide nanoplate to a sample of interest, the nanoplate being attached to a dye; measuring or identifying an emission of the sample; and determining a target property based on the measured or determined emission. The emission can include, for example, a fluorescent emission. The target property can include, for example, one or more of an amount or quantity of the target, a location of the target, a presence of the target, a shape or size of the target, and the like. The method can include introducing a plurality (e.g., a stack of nanoplates) of said metal oxide nanoplates to the sample, each nanoplate including at least one dye-attached surface. Further, the nanoplates can be at least partly surrounded by at least one polymer. The polymer can be, for example, hydrophobic, hydrophilic, or both. The polymer can be a block co-polymer. The metal oxide can include one or more of La₂O₃, Pr₂O₃, Nd₂O₃, Sm₂O₃, Gd₂O₃, Dy₂O₃, Ce₂O₃, Tb₂O₃, Er₂O₃, Eu₂O₃, Lu₂O₃, Tm₂O₃, Ho₂O₃, Pm₂O₃, and Yb₂O₃. In some aspects the metal oxide can include lanthanide oxide. The target can include, for example, one or more of an organ, a tissue, a cell, an antigen, a receptor, a protein, an enzyme, an oligopeptide, a polypeptide, a nucleobase, a nucleic acid molecule, a liposome, a ligand, a biomolecule, an antibody, a monoclonal antibody, a natural or synthetic drug, a synthetic polymer, a hormone, a lymphokine, a cytokine, a toxin, a hapten, a carbohydrate, a sugar and the like. The nanoplate(s) can be attached to a component, for example, one or more of a biomolecule, an antibody, an aptamer, an antigen, a monoclonal antibody, a protein, an enzyme, a receptor, a natural or synthetic drug, a synthetic polymer, a hormone, a lymphokine, a cytokine, a toxin, a ligand, a hapten, a carbohydrate, a sugar, an oligopeptide, a polypeptide, a nucleobase, a nucleic acid molecule, a liposome, and the like. In some aspects the emission can result from the component contacting the target.

In some aspects, there can be methods of making a labeling reagent. The methods can include, for example, attaching a metal oxide nanoplate to a dye; optionally dispersing the dye-attached nanoplates in an organic solvent; combining the nanoplate solution with an amphiphilic block-co-polymer solution, such that the nanoplates are at least partly surrounded by the co-polymer; and obtaining the dye-attached, co-polymer-surrounded nanoplate. The methods optionally include dispersing an amphiphilic block-co-polymer in water.

In some aspects, there can be methods of making a reagent. The methods can include, for example, attaching a metal oxide nanoplate to a dye; optionally dispersing the dye-attached nanoplates in an organic solvent; optionally dispersing an amphiphilic block-co-polymer in water; combining the nanoplate solution with the amphiphilic block-co-polymer solution, such that the nanoplates are at least partly surrounded by the co-polymer; attaching at least one of the dye-attached, co-polymer-surrounded nanoplate to a component, for example, one or more of a biomolecule, an antibody, an aptamer, an antigen, a monoclonal antibody, a protein, an enzyme, a receptor, a natural or synthetic drug, a synthetic polymer, a hormone, a lymphokine, a cytokine, a toxin, a ligand, a hapten, a carbohydrate, a sugar, an oligopeptide, a polypeptide, a nucleobase, a nucleic acid molecule, a liposome, and the like.

In some aspects, there can be methods of preparing reagent. The methods can include, for example, attaching a dye to at least one surface of a metal oxide nanoplate; and linking the dye-attached nanoplate to a component, for example, one or more of a biomolecule, an antibody, an aptamer, an antigen, a monoclonal antibody, a protein, an enzyme, a receptor, a natural or synthetic drug, a synthetic polymer, a hormone, a lymphokine, a cytokine, a toxin, a ligand, a hapten, a carbohydrate, a sugar, an oligopeptide, a polypeptide, a nucleobase, a nucleic acid molecule, a liposome, and the like. The methods further can include attaching the dye to a surface of each of a plurality of metal oxide nanoplates. The methods further can include assembling the plurality of dye-attached nanoplates into a stack of nanoplates, which optionally can be configured to self-assemble into a stack of nanoplates. The plurality of nanoplates can include an interface between adjacent nanoplates, for example a dye layer between plates. The methods further can include at least partly surrounding the nanoplate or plurality of the nanoplates with at least one polymer as described herein. The surface density of the dye can be any desired density, for example, the density can be at least about 1 dye molecule per square nanometer of area of the surface, about 5 dye molecules per square nanometer of area of the surface, or from about 1 to 5 dye molecules per square nanometer of area of the surface, for example. In some aspects the metal oxide can be one or more of La₂O₃, Pr₂O₃, Nd₂O₃, Sm₂O₃, Gd₂O₃, Dy₂O₃, Ce₂O₃, Tb₂O₃, Er₂O₃, Eu₂O₃, Lu₂O₃, Tm₂O₃, Ho₂O₃, Pm₂O₃, Yb₂O₃, lanthanide oxide, and the like.

In some aspects, provided are methods of imaging, including bio-imaging. The methods can include contacting a material, such as a biological tissue with a reagent as described herein; and detecting an emission from the material.

Some aspects relate to methods of detecting a target within a biological material. The methods can include, for example, contacting a reagent as described herein with a biological material; and detecting an emission from the dye in the reagent. For example, the target can include, for example, a biomolecule, a cell, a nucleic acid, an antigen, an antibody, an aptamer, a protein, an enzyme, a receptor, a natural or synthetic drug, a synthetic polymer, a hormone, a lymphokine, a cytokine, a toxin, a ligand, a hapten, a carbohydrate, a sugar, an oligopeptide, a polypeptide, a nucleobase, a nucleic acid molecule, a liposome, and the like.

Some aspects relate to methods of bio-imaging surgery. The methods can include, for example, the detecting of a reagent as described herein in a tissue and performing a surgical procedure on some part of the tissue or near the tissue where the reagent was detected. The reagent can be detected based upon the emission of a dye signal.

The foregoing is a summary and thus contains, by necessity, simplifications, generalization, and omissions of detail; consequently, those skilled in the art will appreciate that the summary is illustrative only and is not intended to be in any way limiting. Other aspects, features, and advantages of the devices and/or processes and/or other subject matter described herein will become apparent in the teachings set forth herein. The summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 shows an illustrative embodiment in which a labeling reagent comprises a plurality of nanoplates.

FIG. 2 shows an illustrative embodiment in which dye molecules are attached to nanoplate-interface surfaces and to nanoplate-non-interface surfaces

FIG. 3 shows a cross section of an illustrative embodiment of a reagent 300 including a stack of nanoplates, surrounded by a surrounding layer.

FIG. 4 shows an illustrative embodiment of a process for making a labeling reagent.

FIG. 5 shows an illustrative embodiment of a process for determining a target property using a nanoplate reagent.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

Organic dyes and quantum dots are used for various applications, including for example, bio-imaging. While the cadmium (Cd) chalcogenide semiconductor quantum dots emit very strong light, the toxicity of Cd can be undesirable in some applications. The organic dyes can suffer so-called photobleaching in some applications, for example, when used in physiological conditions.

Embodiments herein relate to reagents, which reagents can provide desirable light emitting characteristics with decreased toxicity and/or less photobleaching.

The reagents can include one or more metal oxide nanoplates and one or more dyes attached to a surface of the one or more nanoplates. The reagents can provide a large surface area to which the dye can attach such that a very large quantity of dye molecules can be used. As more dye molecules are attached to the reagent, the reagent may be able to emit a more intense dye signal. The reagents farther can include a coating at least partially surrounding or encapsulating the one or more nanoplates and one or more attached dyes. The reagents also can include additional components, such as, for example, antibodies, antigens, ligands, receptors, nucleic acids, other chemical moieties, components of the same, and others as described elsewhere herein. The reagents can be delivered to or contacted with a target, whereupon the dye molecules can be liberated or exposed so that a dye signal can be emitted.

The term “dye,” as used herein refers to any reporter group whose presence can be detected by its light absorbing or light emitting properties. The term “dye” encompasses, for example, fluorescent compounds. Fluorescent dyes may, for example, include fluorescein-type dyes, rhodamine-type dyes, cyanine-type dyes, and energy-transfer dye pairs. In some instances, a dye can include a label or tag.

The term “nanoparticle,” as used herein, refers to a particle in which one, more than one or all dimensions are less than about 1000, 500, 300, 100, 50, 30, 10, 5, 3, or 1 nm in length. In some instances, each dimension of the nanoparticles is less than about 1000, 500, 300, 100, 50, 30, 10, 5, 3, or 1 nm in length. The reagents described and made according to the methods described herein, for example the nanoplates and nanoplate stacks, (coated, uncoated, and with or without components), can be referred to as nanoparticles in some embodiments.

The term “nanoplate,” as used herein, refers to a nanoparticle characterized by lengths along a first, second and third dimension, the first dimension length being longer than or equal to the second dimension length, and the second dimension length be longer than the third dimension length, wherein the ratio of the second dimension length to the third dimension length is at least about 3:1, 4:1, 5:1, 7:1, 10:1, 15:1, 20:1 or 50:1. In some instances, an aspect ratio of the nanoplate can be, for example, at least about 3:1, 4:1, 5:1, 7:1, 10:1, 15:1, 20:1 or 50:1.

### Reagents and Methods of Making the Same

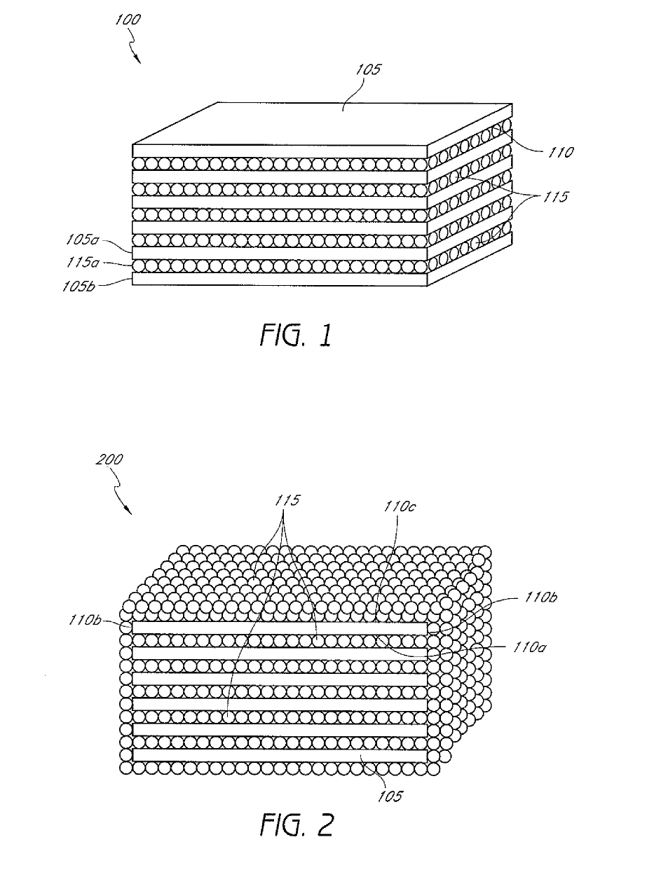

FIG. 1 shows an embodiment in which a labeling reagent 100 includes one or more nanoplates 105 (also 105a and 105b). One or more surfaces 110 of the one or more of the nanoplates 105 may attach to a dye (e.g., dye molecules 115). In some embodiments, a configuration of a plurality (that is, more than one) of said nanoplates 105 (e.g., in a stack) and/or a configuration of a nanoplate relative to another structure (e.g., a surrounding layer) is such to effectively attach the dye to one or more of the nanoplates 105. For example, two or more nanoplates 105 may apply at least partially opposing forces on a dye molecule 115. In another example, two or more nanoplates 105 can restrict movement of a dye molecule 115 along, for example, at least one dimension. In the embodiment of FIG. 1, dye molecules 115 are effectively “sandwiched” between the nanoplates 105.

A dye-attached surface 110 may include an interface between adjacent nanoplates 105. For example, referencing FIG. 1, the “interface” between nanoplates 105a and 105b is dye layer 115a. In some instances the surfaces 110 of adjacent nanoplates 105 can be connected via a dye, dye molecule or dye layer, such that the nanoplates 105a and 105b are not in direct contact but are instead indirectly in contact via the dye, dye molecule or dye layer. In some instances, the dye, dye molecule or dye layer connects adjacent nanoplates 105 and is in direct contact with two adjacent nanoplates 105a and 105b. In other instances, the connection is formed using the dye, dye molecules or dye layer and one or more other intermediate structures. The dye layer may or may not be a continuous layer and/or may include, for example, a plurality of dye molecules 115.

The nanoplate 105 may be comprised of a metal oxide, such as, for example, La₂O₃, Pr₂O₃, Nd₂O₃, Sm₂O₃, Gd₂O₃, Dy₂O₃, Ce₂O₃, Tb₂O₃, Er₂O₃, Er₂O₃, Eu₂O₃, Lu₂O₃, Tm₂O₃, Ho₂O₃, Pm₂O₃, and/or Yb₂O₃. The nanoplate 105 may include, for example, a high-k dielectric material (e.g., a material having a dielectric constant greater than or greater than about twice the dielectric constant of silicon dioxide). The nanoplate 105 may include, for example, a lanthanide silicate. The nanoplate 105 may include, for example, a material that is reactive towards acid and/or that has a high affinity towards a carboxyl group. The nanoplate may include, for example, a non-toxic material and/or may be non-toxic.

The nanoplate may include a primary surface characterized by the surface of a side of the nanoplate with the largest or second largest surface area. In some instances, the nanoplate includes two primary surfaces, the primary surfaces being opposite to each other. The nanoplate may include one or more side surfaces. The side surfaces may include surfaces in between two primary surfaces. The side surfaces may be characterized with a surface area less than or equal to surface areas of at least two other sides (primary sides) of the nanoplate.

The nanoplate may include a thickness of for example, at least about, about, or less than about 1 unit cell, 2 unit cells, 3 unit cells, 5 unit cells or 10 unit cells, wherein a unit cell refers to the smallest building block of the nanoplate, such as an atom or molecule. In one embodiment, the thickness of the nanoplate is less than 3 nm. The thickness may be determined based on a size characteristic of a dye molecule. For example, the thickness may be at least about, about or less than about 1/100, 1/10, 1/5, 1/3, ½, ¾, 1, 2, 3, 4, 5 or 10 times that of a dimension of the dye molecule. In some instances, the thickness can be less than and/or equal to that of a dimension (e.g., the largest or smallest dimension) of a dye molecule.

In some instances, a dye can be one that permits association, linking, binding and/or attachment to a surface of a nanoplate 105. A dye may include a carboxyl group (—COOH) group, which may, for example, easily bind to a metal oxide nanoplate surface. For example, —COOH groups can be used with lanthanide metal oxide plates because the lanthanide plates can be very reactive toward acid. Thus the functional group —COOH can react easily and bind to the metal oxide nanoplate surface. A dye may contain other aqueous solubilizing groups, such as sulphonate groups. As used herein, the term “attached” is meant to be construed broadly to include the state of a dye being linked, bound or adhered to a nanoplate, as well as the dye being coated onto, trapped, sandwiched or merely located adjacent to the nanoplate (but not necessarily chemically bound or linked).

The nanoplate 105 may be one formed by a variety of methods, such as that disclosed in Paek et al., Crystal Growth & Design 2007, 7:8, 1378-1380, which is hereby incorporated by reference in its entirety.

In some embodiments, one or more surfaces 110 of a nanoplate 105 can be substantially saturated with dye molecules 115. Dye molecules 115 may be, for example, directly adjacent to or in contact with neighboring dye molecules 115. In some embodiments, one or more surfaces 110 of a nanoplate 105 may be substantially unsaturated with dye molecules 115, for example, 1%-60% of the surface may be covered with dye molecules. Adjacent dye molecules 115 may be separated, e.g., by a space. The surface density of dye molecules on the one or more surfaces may be, for example, greater than about, about, or less than about 1, 10, 100, 500, 1,000, 5,000, 10,000, 50,000, 100,000 or 500,000 dye molecules per square micrometer of area of the surface. The surface density of dye molecules on the one or more surfaces may be, for example, greater than about, about, or less than about 1, 2, 3, 4, 5, 7, 10, 15, 20, 30, 40, 50 or 100 dye molecules per square nanometer of area of the surface. As one example, in some aspects about 1,000 dye molecules can be confined to a 10 nm×10 nm×1 nm surface, such as a metal oxide plate. In some embodiments, a surface density can vary with distance to an edge of the surface 110. For example, the surface density may be higher towards the end of a surface than towards the middle. The surface density of the dye may depend, for example, on a size of the dye molecule 115, a shape of a dye molecule, an absolute or relative concentration of dye molecules 115 in a manufacturing step, a surface-dye attachment time in a manufacturing step, and/or a property of the nanoplate surface 110.

The plurality of nanoplates 105 may be parallel or substantially parallel to each other, as shown in FIGS. 1 and 2. As used herein, “substantially parallel” can mean that the angle between plates can range from about zero degrees to about 5 or about 10 degrees. The plurality of nanoplates 105 may be arranged in a stack. In some instances, each nanoplate 105 can include a surface normal direction, that being a direction normal to the largest surface of the plate. Nanoplates 105 of a stack may be positioned such that a first nanoplate 105 of the stack is offset from a second nanoplate 105 (or from all other nanoplates 105) of the stack in a direction parallel to the surface normal direction. In some instances, edges of one nanoplate in a stack can be aligned with edges of another nanoplate in the stack, while in other embodiments, they are not. Stacks may include nanoplates of the same size or of different sizes. As illustrated in FIG. 1, the nanoplate stack can include dye molecules, or a dye interface, in between the stacked nanoplates. While the embodiments described above focused on nanoplates, it shall be appreciated in view of the present disclosure that other suitable nanomaterials such as nanofibers, nanotubes, nanoparticles, and the like where dye molecules can be attached thereto or placed therein or thereon can be used in lieu of or in addition to the nanoplates.

FIG. 2 depicts an example of an embodiment of a reagent 200 in which dye molecules are attached to more than one surface of a nanoplate. For example, in FIG. 2, dye molecules are attached to nanoplate-interface surfaces 110a, and to nanoplate-non interface surfaces 110b and 110c. Surface 110a is referred to as a nanoplate interface surface because that surface includes a dye molecule interface between surface 110a and the face of the adjacent plate. Dye molecules may attach to at least one side or edge surface 10b of the nanoplate. The side surface 10b may include surfaces of nanoplates having a surface area less than a surface area of at least two other nanoplate surfaces. For example, in the illustration of FIG. 2, dye molecules are shown on two side surfaces 110b, while none are shown on the front side surface/edge and the rear edge is not visible. Dye molecules may attach to at least one non-interface primary surface 110c. The non-interface primary surface 110c (e.g., a surface with the largest or second largest surface area of the nanoplate) may be one that is not facing another nanoplate. In FIG. 2, the non-interface primary surface is the top of the example reagent, for example, where the dye molecules are contacted by only one nanoplate.

The surface may be configured to attract at least one dye molecule or to bond to at least one dye molecule, e.g., based on a structural characteristic of the surface. In some instances, a position of a nanoplate relative to another structure (e.g., a non-nanoplate structure) effectively attaches the dye molecule to the surface. For example, a layer may be positioned next to a side of the stack (e.g., separated by a space, dye molecules or dye layer), such that dye molecules are sandwiched between the side of the stack and the layer.

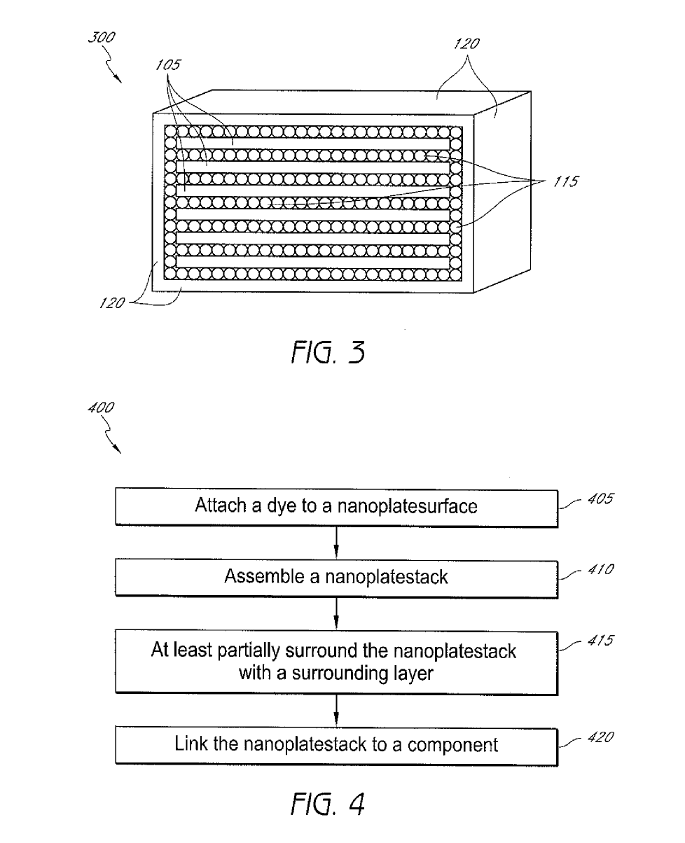

In some instances, the reagents further can include a coating or encapsulation layer. For example, a coating or encapsulation layer can be positioned at least partially around the stack, such that the dye molecules and/or the nanoplate(s) may be at least partially confined within or surrounded by the coating or encapsulation layer. FIG. 3 shows an example of a cross section of a reagent 300 that includes a stack of nanoplates 105 surrounded by a surrounding layer 120. In some embodiments, the surrounding layer 120 may only partially surround a nanoplate or nanoplate stack (e.g., by surrounding a side of the stack or by forming an enclosure with openings or gaps). In some embodiments, at least partially surround or confine can mean that at least about 0%, 5%, 10%, 15%, 20%, 25%, 30%, or 50% and/or less than about 100%, 99.9%, 99.5%, 99%, 95%, 90%, 80% or 70% of the outer surface of the nanoplate/dye molecule stack is covered or surrounded by the coating. In other embodiments, the surrounding layer 120 can fully surround a nanoplate or nanoplate stack. Thus, dye molecules 115 may be trapped or positioned between adjacent nanoplates 105 of the stack and between exterior surfaces of the stack and the surrounding layer 120. In some instances, additional dye molecules 115 can attach to the exterior side (or to a side not facing a nanoplate stack) of the surrounding layer 120. In some instances, a nanoplate stack reagent having the surrounding layer 120 (e.g., reagent 300) can include more dye molecules between nanoplates of the stack than a comparable reagent not including the surrounding layer at least partly due to restriction of dye molecule movement imposed by the surrounding layer.

The surrounding layer may include, or be composed of (without limitation) at least one polymer. The polymer may be, for example, hydrophilic, hydrophobic, or both hydrophilic and hydrophobic. In some instances, the hydrophobic/hydrophilic property of the surrounding layer can be chosen at least partly based on a hydrophobic/hydrophilic property of a dye and/or a hydrophobic/hydrophilic property of a medium into which a reagent having the surround layer will be introduced. For example, a surrounding layer may include a hydrophobic and hydrophilic component when a hydrophobic dye is used and the reagent is to be introduced to a water-based medium. In some instances, (e.g., when the surrounding layer is hydrophilic), the reagent can soluble or can be at least partially water soluble.

In some embodiments, a surrounding layer can include a copolymer or a block copolymer. Polymers and block co-polymers can be selected by the skilled artisan according the particular requirements for the reagents. For example, polymers and/block co-polymers can be selected based upon the environment where the reagent will be used or targeted, etc. Examples of some polymers and co-polymers that can be used include PLGA-PEG, and PCL-PMAA, for example.

The reagents can be designed to react to certain environments, exhibit certain characteristics in certain environments, or to target specific biostructures (e.g., cells). The reagent or a part of the reagent may disintegrate or dissolve within specific biological environments (e.g., low-pH environments, high-pH environments, or environments containing high or low amounts of a particular compound, nutrient, element, transmitter, etc). For example, a surrounding layer may disintegrate or dissolve within a specific biological environment, which can permit dye molecules to be released from the reagent.

For example, certain cancer cells have lower pH than normal cells (J. Am. Chem. Soc. 2007, 129, 5362-5363, which is incorporated herein by reference in its entirety). Thus, the coating on the reagents can include polymers that will selectively dissolve or break down in the pH environment of cancer cells, but not in normal tissue. Therefore, the reagents will expose their dyes and emit a signal in cancerous tissue based upon pH, for example. See for example, B. R. Cho et al. Angew. Chem. Int. Ed. 2008, 47, 2231-2234, which is incorporated herein by reference in its entirety.

In some embodiments, the reagent or a part of the reagent may be attracted toward a specific biostructure. In some embodiments, a specific biological environment or binding to another biostructure can cause a change in the reagent's conformation. For example, the reagent may include calmodulin, which changes its conformation upon binding to calcium. The reagent may include a cameleon calcium indicator. As another example, the reagent may include a FRET- (Fluroresence-Resonance-Energy-Transfer-) based reagent, such as GluSnFR or a derivative thereof, which changes conformation upon binding to a molecule such as glutamate.

While reagents 100, 200 and 300 are shown to have a cubic or block rectangle shape, reagents of any other shapes (e.g., a spherical shape) can be constructed.

In some embodiments, an additional component can be linked to, attached to or can encapsulate a reagent described herein. The component may be or include, for example, a biological unit and/or may be or include a biomolecule, an antibody, an aptamer, an antigen, a monoclonal antibody, a protein, an enzyme, a receptor, a natural or synthetic drug, a synthetic polymer, a hormone, a lymphokine, a cytokine, a toxin, a ligand, a hapten, a carbohydrate, a sugar, an oligopeptide, a polypeptide, a nucleobase, a nucleic acid molecule, and/or a liposome. The component may include a lectin or fragment (or derivative) which retains binding function; a monoclonal antibody (“mAb”, including chimeric or genetically modified monoclonal antibody (e.g., “humanized”)), or fragments or particular domains of antibodies; a peptide; an aptamer; a nucleobase (synthetic, natural, or modified); a nucleic acid molecule (including, but not limited to, single stranded RNA or single-stranded DNA, or single-stranded nucleic acid hybrids); avidin, or streptavidin, or an avidin derivative; and the like. The component may be configured to react with particular biostructures, to particular biological environments, and/or may include a reactive functionality.

FIG. 4 shows an illustrative process 400 for making a labeling reagent. Process 400 begins at step 405, where a dye is attached to a nanoplate surface. In some embodiments, the dye can bind to a nanoplate surface. The dye may be, for example, any dye, including those disclosed herein. The nanoplate surface can be any such surface disclosed herein. For example, the dye may include a carboxyl group and the nanoplate surface may include lanthanide oxide.

At step 410, a nanoplate stack is assembled. In some embodiments, dye-attached nanoplates may self-assemble into a stack. For example, a surface of each of a first and a second nanoplate may be attracted to a dye molecule. In some instances, a concentration of the nanoplates may be high which can promote nanoplate alignment. In some embodiments the dye-attached nanoplates (e.g., before or after stack assembly) can be dispersed in a solvent (e.g., an organic solvent).

At step 415, a surrounding layer can at least partially surround the nanoplate stack. In some instances, nanoplate stacks can be coated by a surrounding layer material. A surrounding layer material may be combined with or may contact a nanoplate medium (e.g., solution) and may then form a layer at least partially surrounding the nanoplate stacks. In one instance, a surrounding layer material (e.g., a block co-polymer) is dispersed in a solvent (e.g., water) and is combined with a nanoplate solution (e.g., an organic solvent-nanoplate solution). At least some of the nanoplates of the nanoplate solution may then be at least partially surrounded by the surrounding layer material, such that at least partly-surrounded dye-attached nanoplate reagents may be obtained.

At step 420, a component can be linked to the nanoplate stack. In some embodiments, this step can include combining the component (e.g., a solution containing the component) and the nanoplate stack (e.g., a solution containing—for example—a surrounded nanoplate stack). The component may be attracted to or have an affinity toward the surrounding layer of the stack, a nanoplate of the stack or a dye of the stack. The component may bind to, attach to, associate with and/or link to a nanoplate stack. (In some instances, the component may bind to, attach to, associate with and/or link to a nanoplate or a dye-attached nanoplate not at least partially surrounded by the surrounding layer.)

### Methods of Using the Nanoplate Materials

The reagents and materials described herein can be used in a variety of applications. In some cases the reagents and materials can simply replace existing “light” or “emission” sources (e.g., dyes, quantum dots, etc.). In some aspects the reagents and materials can be used in bio-imaging, including bio-imaging assisted surgery, cancer detection and treatment, as well as various other applications where light emission can be advantageous, including those that are described herein.

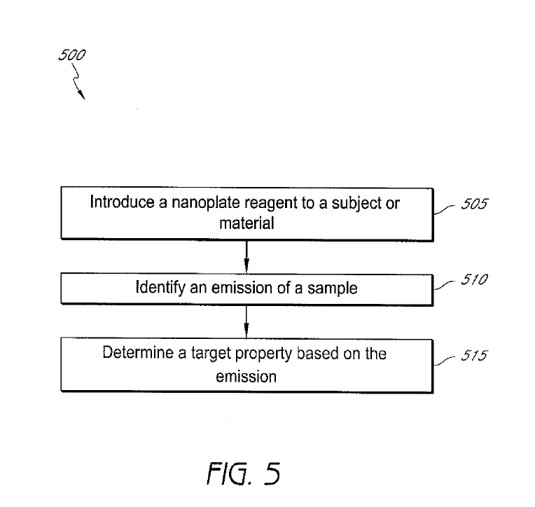

FIG. 5 shows a process 500 for determining a target property using a nanoplate reagent described herein. At step 505, a nanoplate reagent is introduced to or contacted with a subject or material. The nanoplate reagent may include any such reagent disclosed herein. The subject may include but is not limited to, for example, an animal or a mammal, including a human and/or patient. The human and/or patient may be suffering from or at risk of suffering from a condition. The condition may be, for example, cancer or irregular cell growth. The material may include but is not limited to, for example, a biological material (e.g. one or more biological fluids), a tissue, a cell culture and a cell. The material may have been obtained (e.g., through a biopsy) from a subject as described above. For example, a tissue may be obtained from a patient suffering from cancer. Additionally, the reagent can be used with other animals. The reagents can be used for in vitro or in vivo bio-imaging as well as in any other application where a dye or quantum dot may be used, for example.

The nanoplate reagent may be introduced to the subject or material using any appropriate technique. For example, the reagent may be injected into a subject or material, or the reagent may be provided in, for example, an oral, buccal, sublingual, or inhalable form. In some instances, the reagent can be sprayed onto a medium or can be configured to flow over or into the medium such as a patch or a microneedle. Concentration of the active ingredients, e.g., dyes, can be between 1 nanogram to 10 mg per ml (cc).

At step 510, an emission of the sample is identified and/or detected. The emission may be detected using, for example, a camera, a photodiode or a scanner. In some instances, identification of the emission at least partly can indicate that one or more components or reagents have contacted one or more targets (e.g., a target structure or target environment). The target may include, for example, a tissue, a cell, an antigen, a receptor, a protein, an enzyme, an oligopeptide, a polypeptide, a nucleobase, a nucleic acid molecule, a liposome, a ligand, a biomolecule, an antibody, a monoclonal antibody, a natural or synthetic drug, a synthetic polymer, a hormone, a lymphokine, a cytokine, a toxin, a hapten, a carbohydrate, and a sugar. In one embodiment, the component can include or be an antibody and the target can be or include an antigen. In another embodiment, the component can include or be an antigen and the target can be or include an antibody. The target of the reagent may be a biological environment. The biological environment may be, for example, one with a particular pH (e.g., a basic or acidic environment).

In some embodiments, the reagent can be designed so that the emission of the dye does not occur until the reagent reaches a particular location or environment, or until the reagent is altered. For example, an emission of a dye initially may be at least partially blocked by the surrounding layer. Upon administration or delivery of the reagent, the component of nanoplate reagent may then bind to, attach to, link to or associate with a target structure. For example, an antibody component may bind to an antigen target structure. This binding, attachment, linking or association can change a property of the reagent. For example, a conformation of the reagent may change, or part of the reagent may dissociate from another part of the reagent. For example, the surrounding layer may be at least partially degraded. In other instances, an environmental condition in the target area or region (e.g., a pH) may at least partially degrade the surrounding layer, independent of or in addition to the action of the component. Thus, the target environment and/or a component of a reagent binding to, attaching to, linking to or associating with the target structure may influence (e.g., occurrence of or amount of) a signal (e.g., fluorescent light) emitted by a dye within the reagent. In still other instances, a target structure (e.g., an enzyme) may at least partially degrade the surrounding layer.

Dye molecules may thus be exposed to an external environment and may move or diffuse away from the nanoplates. The dye molecules may emit an (e.g., fluorescent) emission, which may now be detectable since, for example, the surrounding layer no longer at least partially blocks or prevents the emission.

In some embodiments, the nanoplate reagent continuously or semi-continuously emits an emission. In some embodiments, an emission can be triggered by an event. For example, the emission may be triggered by illumination by a light source and/or electromagnetic energy.

At step 515, a target property is determined based at least partly on the emission. The target property may include, for example, a presence, an amount and/or a location of the target. In one instance, an existence of an emission may indicate that a target structure, region or environment is present within the medium or sample. In another instance, an intensity of an emission at least partly indicates a target property, such as a predicted amount of a target structure within a medium or subject or part of the subject. In yet another instance, the position of the emission at least partly can indicate a target property, such as a predicted location of a target structure or environment. An emission at least partly indicates a biological environment characteristic. For example, an emission may at least partly indicate a predicted pH or range of pHs within a medium, subject or part of the medium or subject.

In some embodiments, an emission may at least partially indicate a concentration of a particular protein or other component in a system. If the number of reactive groups on a protein which can react with a component probe is known, the fluorescence per molecule can be known and the concentration of these molecules in the system can be determined by the total fluorescence intensity of the system. This particular method can be used to measure the concentration of various labeled analytes using microtitre plate readers or other known immunofluorescence detection systems. The concentration of fluorescently labeled material can also be determined using, for example, fluorescence polarization detection instruments.

The determination of the target property may include characterizing an activity of a biostructure or region. For example, emission may at least partially indicate that a neuron is firing action potentials or that a region (e.g., of the brain) is exhibiting high or higher firing rates than a threshold or baseline. Such may be indicated, for example, by an emission from a reagent sensitive to a neurotransmitter concentration (e.g., a calmodulin-containing or FRET-based reagent) indicating the state of one or more receptors (e.g., open or closed) associated with the neurotransmitter or indicating a concentration (e.g., an intra-cellular or extra-cellular concentration) of the neurotransmitter.

The determination of the target property may further include a diagnosis or prognosis. For example, an emission (e.g., of a particular intensity and/or at a particular location) may at least partially indicate that a subject is suffering from or is likely suffering from cancer. The emission may indicate that a prognosis is improving or worsening (e.g., that a tumor is growing or shrinking).

Furthermore, the determination of the target property can include imaging a tissue or structure, which can be, for example, operated on, surgically treated or removed, modified, or the like.

The embodiments are explained herein by way of example, and there are numerous modifications, variations and other embodiments that may be employed that would still be within the scope of the present disclosure. Components can be added, removed, and/or rearranged. Additionally, processing steps may be added, removed, or reordered. For example, in some embodiments, process 500 does not include step 515 and/or process 400 does not include step 420. A wide variety of designs and approaches are possible.

For purposes of this disclosure, certain aspects, advantages, and novel features of certain embodiments are described herein. It is to be understood that not necessarily all such advantages may be achieved in accordance with any particular embodiment. Thus, for example, those skilled in the art will recognize that the embodiment may be embodied or carried out in a manner that achieves one advantage or group of advantages as taught herein without necessarily achieving other advantages as may be taught or suggested herein.

The herein described subject matter sometimes illustrates different components contained within, or connected with, different other components. It is to be understood that such depicted architectures are merely illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A reagent comprising:
one or more metal oxide nanoplates; and
one or more dyes attached to surface of said one or more metal oxide nanoplates.

2. The reagent of claim 1, further comprising a plurality of said metal oxide nanoplates, each nanoplate comprising at least one dye-attached surface.

3. The reagent of claim 2, wherein the at least one dye-attached surface includes interfaces between adjacent nanoplates.

4. The reagent of claim 2, wherein the one or more nanoplates includes a stack of said nanoplates.

5. The reagent of claim 2, further comprising at least one polymer at least partly surrounding the plurality of said nanoplates.

6. The reagent of claim 5, wherein the at least one polymer is hydrophilic, hydrophobic, or both hydrophobic and hydrophilic.

7. The reagent of claim 5, wherein the at least one polymer includes a block co-polymer.

8. The reagent of claim 1, wherein a thickness of the nanoplate is less than 3 nm.

9. The reagent of claim 1, wherein the dye is configured to link to the surface of the nanoplate.

10. The reagent of claim 1, wherein the dye includes a carboxyl group.

11. The reagent of claim 1, wherein the surface density of the dye is at least about 1 dye molecule per square nanometer of area of the surface or is at least about 5 dye molecules per square nanometer of area of the surface.

12. The reagent of claim 1, wherein the metal oxide includes at least one of La₂O₃, Pr₂O₃, Nd₂O₃, Sm₂O₃, Gd₂O₃, Dy₂O₃, Ce₂O₃, Tb₂O₃, Er₂O₃, Eu₂O₃, Lu₂O₃, Tm₂O₃, Ho₂O₃, Pm₂O₃, and Yb₂O₃.

13. The reagent of claim 1, wherein the reagent is water soluble.

14. The reagent of claim 1, further comprising a component attached to the reagent.

15. The reagent of claim 14, wherein the component includes a biological unit.

16. The reagent of claim 14, wherein the component includes at least one of a biomolecule, an antibody, an aptamer, an antigen, a monoclonal antibody, a protein, an enzyme, a receptor, a natural or synthetic drug, a synthetic polymer, a hormone, a lymphokine, a cytokine, a toxin, a ligand, a hapten, a carbohydrate, a sugar, an oligopeptide, a polypeptide, a nucleobase, a nucleic acid molecule, and a liposome.

17. The reagent of claim 14, wherein the component includes an antibody.

18. A method of bio-imaging, comprising:
contacting a biological tissue with a reagent of claim 1; and
detecting an emission from the tissue.

19. A method of detecting a target within a biological material, comprising:
contacting a reagent of claim 1 with a biological material; and
detecting an emission from the dye in the reagent.

20. The method of claim 19, wherein the target is selected from the group consisting of a biomolecule, a cell, a nucleic acid, an antigen, an antibody, an aptamer, a protein, an enzyme, a receptor, a natural or synthetic drug, a synthetic polymer, a hormone, a lymphokine, a cytokine, a toxin, a ligand, a hapten, a carbohydrate, a sugar, an oligopeptide, a polypeptide, a nucleobase, a nucleic acid molecule, and a liposome

21. A method of preparing a reagent, the method comprising:
attaching a dye to at least one surface of a metal oxide nanoplate; and
linking the dye-attached nanoplate to a component.

22. The method of claim 21, further comprising attaching the dye to a surface of each of a plurality of metal oxide nanoplates.

23. The method of claim 22, further comprising assembling the plurality of dye-attached nanoplates into a stack of nanoplates.

24. The method of claim 22, wherein the dye-attached nanoplates are configured to self-assemble into a stack of nanoplates.

25. The method of claim 22, wherein the at least one dye-attached surface includes interfaces between adjacent nanoplates.

26. The method of claim 22, further comprising at least partly surrounding the plurality of said nanoplates with at least one polymer.

27. The method of claim 26, wherein the at least one polymer is hydrophilic, hydrophobic, or both hydrophobic and hydrophilic.

28. The method of claim 26, wherein the at least one polymer includes a block co-polymer.

29. The method of claim 21, wherein a thickness of the nanoplate is that of one unit cell.

30. The method of claim 21, wherein the dye includes a carboxyl group.

31. The method of claim 21, wherein the surface density of the dye is at least between about 1 dye molecule to about 5 dye molecules per square nanometer of area of the surface.

32. The method of claim 21, wherein the metal oxide includes at least one of La₂O₃, Pr₂O₃, Nd₂O₃, Sm₂O₃, Gd₂O₃, Dy₂O₃, Ce₂O₃, Tb₂O₃, Er₂O₃, Eu₂O₃, Lu₂O₃, Tm₂O₃, Ho₂O₃, Pm₂O₃, and Yb₂O₃.

33. The method of claim 21, further comprising at least partly surrounding the dye-attached nanoplate with at least one polymer.

34. The method of claim 33, wherein the at least one polymer is hydrophilic, hydrophobic, or both hydrophobic and hydrophilic.

35. The method of claim 33, wherein the at least one polymer includes a block co-polymer.

36. The method of claim 21, wherein the component includes a biological unit.

37. The method of claim 21, wherein the component includes at least one of a biomolecule, an antibody, an aptamer, an antigen, a monoclonal antibody, a protein, an enzyme, a receptor, a natural or synthetic drug, a synthetic polymer, a hormone, a lymphokine, a cytokine, a toxin, a ligand, a hapten, a carbohydrate, a sugar, an oligopeptide, a polypeptide, a nucleobase, a nucleic acid molecule, and a liposome.

38. The method of claim 21, wherein the component includes an antibody.

39. The method of claim 21, wherein the component includes a reactive functionality.

40. The method of claim 21, wherein the linking includes attaching the dye-attached nanoplate to the component.

41. The method of claim 21, wherein the linking includes encapsulating the dye-attached nanoplate within the component.

42. The method of claim 21, further comprising administering the linked nanoplate to a subject.

43. The method of claim 21, further comprising introducing the linked nanoplate to a biological material.

44. The method of claim 43, wherein the biological material includes at least one of a tissue, a cell culture and a cell.

45. A method for determining a property of a target, the method comprising:
introducing a metal oxide nanoplate to a sample of interest, the nanoplate being attached to a dye;
measuring an emission of said sample; and
determining a target property based on said measured emission.

46. The method of claim 45, wherein said emission includes fluorescent emission.

47. The method of claim 45, wherein the target property includes an amount of the target.

48. The method of claim 45, wherein the target property includes a location of the target.

49. The method of claim 45, wherein the target property includes a presence of the target.

50. The method of claim 45, comprising introducing a plurality of said metal oxide nanoplates to the sample, each nanoplate including at least one dye-attached surface.

51. The method of claim 50, wherein the plurality of said nanoplates includes a stack of said nanoplates.

52. The method of claim 50, wherein the at least one dye-attached surface includes interfaces between adjacent nanoplates.

53. The method of claim 50, wherein at least one polymer at least partly surrounds the plurality of nanoplates.

54. The method of claim 53, wherein the at least one polymer is hydrophilic, hydrophobic, or both hydrophobic and hydrophilic.

55. The method of claim 53, wherein the at least one polymer includes a block co-polymer.

56. The method of claim 45, wherein a thickness of the nanoplate is that of one unit cell.

57. The method of claim 45, wherein the dye includes a carboxyl group.

58. The method of claim 45, wherein the surface density of the dye is in the range of at least about 1 dye molecule to about 5 dye molecules per square nanometer of area of the surface.

59. The method of claim 45, wherein the metal oxide includes at least one of La₂O₃, Pr₂O₃, Nd₂O₃, Sm₂O₃, Gd₂O₃, DY₂O₃, Ce₂O₃, Tb₂O₃, Er₂O₃, Eu₂O₃, Lu₂O₃, Tm₂O₃, Ho₂O₃, Pm₂O₃, and Yb₂O₃.

60. The method of claim 45, wherein the dye-attached nanoplate is at least partly surrounded by a polymer.

61. The method of claim 60, wherein the at least one polymer is hydrophilic, hydrophobic, or both hydrophobic and hydrophilic.

62. The method of claim 60, wherein the at least one polymer includes a block co-polymer.

63. The method of claim 45, wherein the target includes at least one of a cell, an antigen, a receptor, a protein, an enzyme, an oligopeptide, a polypeptide, a nucleobase, a nucleic acid molecule, a liposome, a ligand, a biomolecule, an antibody, a monoclonal antibody, a natural or synthetic drug, a synthetic polymer, a hormone, a lymphokine, a cytokine, a toxin, a hapten, a carbohydrate, and a sugar.

64. The method of claim 45, wherein said nanoplate is attached to a component.

65. The method of claim 64, wherein the component includes at least one of a biomolecule, an antibody, an aptamer, an antigen, a monoclonal antibody, a protein, an enzyme, a receptor, a natural or synthetic drug, a synthetic polymer, a hormone, a lymphokine, a cytokine, a toxin, a ligand, a hapten, a carbohydrate, a sugar, an oligopeptide, a polypeptide, a nucleobase, a nucleic acid molecule, and a liposome.

66. The method of claim 64, wherein said emission is a result of said component contacting said target.

67. The method of claim 64, wherein the target includes one of an antibody and an antigen and the component includes the other of an antibody and an antigen.

68. A method of making a labeling reagent for labeling a target comprising:
attaching a metal oxide nanoplate to a dye;
combining the nanoplate attached to a dye with an amphiphilic block-co-polymer solution, such that the nanoplate attached to a dye is at least partly surrounded by the co-polymer; and
obtaining the dye-attached, co-polymer-surrounded nanoplate.

69. The method of claim 68, further comprising one or both of dispersing the dye-attached nanoplates in an organic solvent and dispersing an amphiphilic block-co-polymer in water.

70. A method of making a labeled component comprising:
attaching a metal oxide nanoplate to a dye;
combining the nanoplate attached to a dye with an amphiphilic block-co-polymer solution, such that the nanoplate attached to a dye is at least partly surrounded by the co-polymer; and
attaching at least one dye-attached, co-polymer-surrounded nanoplate to a component.

71. The method of claim 70, wherein the component includes at least one a biomolecule, an antibody, an aptamer, an antigen, a monoclonal antibody, a protein, an enzyme, a receptor, a natural or synthetic drug, a synthetic polymer, a hormone, a lymphokine, a cytokine, a toxin, a ligand, a hapten, a carbohydrate, a sugar, an oligopeptide, a polypeptide, a nucleobase, a nucleic acid molecule, and a liposome.

72. The method of claim 70, further comprising one or more of dispersing the dye-attached nanoplates in an organic solvent, dispersing an amphiphilic block-co-polymer in water, and obtaining the dye-attached, co-polymer-surrounded nanoplate.
